(19) United States
(12) Patent Application Publication
(10) Pub. No.: US 2010/0209947 A1
(43) Pub. Date: Aug. 19, 2010

(54) Free human serum IgE immunoenzymetric assay and methods of use

(75) Inventor: Robert G. Hamilton, Baldwin, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/985,350

(22) Filed: Nov. 13, 2007

Related U.S. Application Data
(63) Continuation of application No. PCT/US06/18873, filed on May 15, 2006.
(60) Provisional application No. 60/680,887, filed on May 13, 2005.

(51) Int. Cl. G01N 33/53; G01N 33/566

(52) U.S. Cl. 435/7.92; 435/7.1; 436/501; 435/7.9

(57) ABSTRACT

The present invention relates to the development of novel of free blood fluid IgE Immunoenzymetric assay that is specifically designed to evaluate the blood fluid of patients on therapeutic agents designed to reduce free serum IgE levels, such as Omalizumab. The assay displays the robustness required for clinical analysis of serum containing such agents as Omalizumab.

### CROSS REFERENCE TO RELATED APPLICATION

This application claims priority to U.S. Provisional Patent Application Ser. No. 60/680,887 filed May 13, 2005, the entire disclosure of which is incorporated herein by reference in its entirety.

### FIELD OF THE INVENTION

The present invention relates to novel immunoassay for quantitative measurement of free blood fluid level IgE antibody in patients receiving a therapeutic agent for reducing free IgE blood fluid levels.

### BACKGROUND OF THE INVENTION

Human allergic disease has been managed using a combination of allergen avoidance, pharmacotherapy and immunotherapy (Naclerio, 1991; Norman 2004). In 2003, Omalizumab, a recombinant humanized IgG1 monoclonal anti-human IgE Fc (Xolair®), was licensed for use in the United States to treat moderate to severe persistent allergic asthma. Omalizumab binds circulating IgE, blocking IgE binding to alpha chain of FcεR1 receptors and down regulating the number of FcεR1 on mast cells and basophils (Chang et al, 1990; Saini et al. 1999; Davis, 2004). Reduced cell bound IgE can result in a concomitant reduction in mediator release and reduced allergy symptoms (Holgate et al 2004; Lin et al. 2004). The best clinical results have been observed in asthma patients receiving 0.016 mg of Omalizumab per kg per KIU/L of IgE for a minimum duration of 12 weeks (Bousquet et al. 2004).

Unfortunately, not all asthma patients receiving Omalizumab experience the desired clinical benefits of reduced usage of their rescue or controller medications, improved lung function, decreases in asthma exacerbation rates and/or enhanced quality of life. In these patients, the treating physician needs to know whether the prescribed Omalizumab dose has reduced free serum IgE levels into the therapeutic range (>95%). To assess this issue, a serological measurement of non-Omalizumab complexed “free” IgE is needed, since the level of free IgE in serum identifies patients who may have been under dosed and in whom non-complexed IgE is free to continue to arm FcεR1 on mast cells and basophils. Presently, no CLIA-88 licensed clinical laboratory measures free human IgE because documented serological assays have not been available. It would be highly desirable to develop a sensitive quantitative assay measuring free serum IgE levels in patients receiving therapeutic agents which are administered to patients for the purpose of reducing free IgE serum levels, and consequently monitor the effectiveness of the therapeutic agent.

### SUMMARY OF THE INVENTION

The present invention relates to the development of novel methods and assays for the quantitative measurement of free blood fluid IgE, more specifically designed to evaluate the blood fluid of patients treated with therapeutic agents designed to reduce free blood fluid IgE levels, such as Omalizumab.

More specifically, the present invention relates to a method of quantifying the free blood fluid IgE level in a patient comprising the steps of a) contacting the patient's blood fluid with an IgE directed antibody immobilized on a substrate under conditions suitable for formation of a IgE:capture antibody complex; b) contacting the substrate of step (a) with a detection labeled FcεR1α receptor under conditions suitable for formation of an IgE: FcεR1α receptor complex; and c) determining the quantity of free blood fluid IgE by measuring the amount of detection label in comparison to known quantity level standards for IgE.

In a preferred embodiment, capture antibody is a monoclonal antibody, and in particular,

wherein the monoclonal antibody binds to the Fc portion of the IgE and does not bind to an epsilon heavy chain antigenic determinant bound by the α-chain of the FcεR1 receptor. In particular embodiments, the capture antibody is the monoclonal murine IgM anti-human IgE Fc clone HP6061 or the monoclonal murine IgG1 anti-human IgE Fc clone HP6029. In still other embodiments, the patient's blood fluid is comprised of serum or plasma and other human body fluids such as tear fluids, lavages of nasal and bronchial passages.

Further other embodiments include the FcεR1α receptor protein (i.e., secreted extracellular domain of the alpha chain) comprising at least a portion of a human Fc-ε-R alpha chain that binds IgE, particularly from the FcεR1α receptor comprising the amino acid sequence of SEQ ID No. 1. It is encoded by SEQ ID NO: 2. Specifically, the amino acid sequence stretches from the 1st amino acid after the signal sequence through amino acid 172 (where amino acid 1 is the 1st amino acid after the signal sequence).

The FcεR1α receptor can be conjugated to a detection label selected from the group consisting of a radioactive label, a fluorescent label, a chemiluminescent label, a chromophoric label and a ligand or more particularly, a detection label selected from the group consisting of fluorescein, a radioisotope, a phosphatase, biotin, biotin-related compounds, avidin, avidin-related compounds and a peroxidase.

In another preferred embodiment of the present invention, the substrate comprises a material selected from the group consisting of plastic, glass, gel, celluloid, paper, particulate material and latex, polystyrene, nylon, nitrocellulose, agarose and magnetic resin. Furthermore, the substrate can comprise a shape selected from the group consisting of a well, a plate, a dipstick, a bead, a lateral flow apparatus, a membrane, a filter, a tube, a dish, a celluloid-type matrix and a magnetic particle. Such substrates can be made as an ELISA plate, a dipstick, a radioimmunoassay plate, agarose beads, plastic beads, latex beads, immunoblot membranes and immunoblot papers.

The present invention also relates to quantitative determination of free blood fluid IgE levels performing assays selected from the group consisting of enzyme-linked immunoassays, radioimmunoassays, immunoprecipitations, fluorescence immunoassays, chemiluminescent assay, immunoblot assays, lateral flow assays, agglutination assays and particulate-based assays.

Another embodiment of the present invention includes a method of determining the efficacy of a therapeutic agent that lowers a patient's free blood fluid IgE level by quantifying the patient's free blood fluid IgE levels before and after administration of the therapeutic agent comprising the steps of: a) contacting the patient's blood fluid with an IgE directed antibody immobilized on a substrate under conditions suitable for formation of a IgE:capture antibody complex; b) contacting the substrate of step (a) with a detection labeled FcεR1α receptor under conditions suitable for formation of an IgE: FcεR1α receptor complex; c) determining the quantity of free blood IgE by measuring the amount of detection label in comparison to known quantity level standards for IgE; d) comparing the patient's free blood fluid IgE levels before and after administration of the therapeutic agent. In a preferred embodiment, the patient's blood fluid is serum, capture antibody binds to the Fc portion of the IgE and does not bind to an antigenic determinant of the α-chain of the FcεR1α receptor and the therapeutic agent is omalizumab.

The present invention also includes a kit for performing methods of the present invention. In particular, the invention relates to an assay kit for quantifying the free blood fluid IgE level in a patient comprising an IgE capture antibody and a detection labeled FcεR1α, receptor.

Other aspects of the invention are discussed infra.

### BRIEF DESCRIPTION OF THE FIGURES

FIG. 1: The free IgE IEMA 5 point calibration curve (open circles: 1000, 200, 50, 20, 2 kIU/L) and 10 point calibration curve (closed circles: 1000, 500, 250, 125, 62.5, 31.25, 15.625, 7.813, 3.906, 1.953 kIU/L) that were analyzed in the same assay. The assay demonstrates parallelism as shown by results of the two dilutions of low IgE control (closed triangle; inter-dilutional % CV=3.7%) and a test serum (open triangle, inter-dilutional % CV=3.0) that contained free IgE levels close to the analytical sensitivity of the assay.

FIG. 2. Precision profile analysis of the free serum IgE IEMA. The intra-assay coefficient of the variation (CV) of the interpolated dose (Y-axis) is plotted against the dose (X-axis). The working range of the assay is defined from 10 kIU/L (twice the negative control binding) to 1000 kIU/L. Over the working range of the IEMA, intra-assay precision is <15% (n=180 duplicate measurements collected from 6 IEMAs).

FIG. 3: Top Panel: Regression analysis of free serum IgE (Y axis) versus total serum IgE (X-axis) levels as measured by IEMA in serum from 33 adults with a spectrum of total serum IgE levels prior to Omalizumab administration [r²=0.98, Y=1.06X−0.05, n=33]. Bottom panel: Regression analysis of the total serum IgE as measured in the same sera in the Pharmacia ImmunoCAP 250 (Y axis) and IEMA (X axis; bottom panel) [r²=0.99, Y=0.90X+0.32, n=33].

FIG. 4. Inhibition curves displaying the percentage reduction in free human IgE in 6 sera with IgE concentrations ranging from 55.7 to 240 kIU/L that had been incubated with increasing concentrations of Omalizumab to produce [humanized IgG1 anti-human IgE Fc] to [human IgE] molar ratios from 0.22 to 9500. Reductions of ≧90% were observed at anti-IgE to IgE ratios from 2 to 200, indicating heterogeneity. The IgE concentrations of the sera in the absence of Omalizumab were 55.7 kIU/L (open triangles), 121.2 kIU/L (closed triangles), 236 kIU/L (closed circles), 222 kIU/L (closed squares), 73.3 kIU/L (open circles) and 240 kIU/L (open boxes).

FIG. 5. Free (close symbols) and total (open symbols) serum IgE levels (kIU/L) measured in the free and total serum IgE IEMAs (Y-axis), respectively, for two sera (circles and triangles) in the absence and presence of Omalizumab from 100 ng/ml to 1 mg/ml (X-axis). No significant change in the total serum IgE levels were detected, in spite essentially complete decreases in free IgE levels which indicates that Omalizumab binding to IgE does not interfere in the total serum IgE IEMA.

FIG. 6 Changes in the total (closed circles-solid lines) and free (open circles-dashed lines) IgE for subject 1 (top panel) and subject 2 (bottom panel) as described in Table 1 at baseline, 1 and 3 months post Omalizumab treatment. Subject 1 displayed a 98% decrease in free IgE with increases in measured total serum IgE of 2.5 to 2.8 fold. In contrast, Subject 2 displayed a 50% decrease in free IgE after 3 months of Omalizumab therapy with 3.5 to 4.1 fold increases in total circulating IgE.

FIG. 7 represents the amino acid sequence of the FcεR1α receptor (SEQ ID No.: 1).

FIG. 8 represents the nucleic acid sequence of the FcεR1α receptor (SEQ ID No.: 2).

### DETAILED DESCRIPTION OF THE INVENTION

An estimated 50 million people have allergic disease in America, with approximately 17 million suffering from asthma. Since allergic asthma is an IgE-mediated disease involving the release of vasoactive mediators following the exposure to airborne allergens, reduction of IgE levels on effector cells should have beneficial effects. Unlike other forms of therapy that either treat the symptoms with drugs or modify the immune response following specific allergen immunotherapy, Omalizumab reacts with the binding site on the epsilon heavy chain and effectively blocks the binding of IgE to high affinity cellular receptors on effector cells. The consequence of this complex formation in vivo is a reduction in IgE bound to mast cells and basophils, a concomitant down regulation of FcεR1 receptor numbers on these cells and a reduction in the release of mediators following allergen exposure (Saini et al, 1999; Lin et al, 2004; Soler et al, 2001).

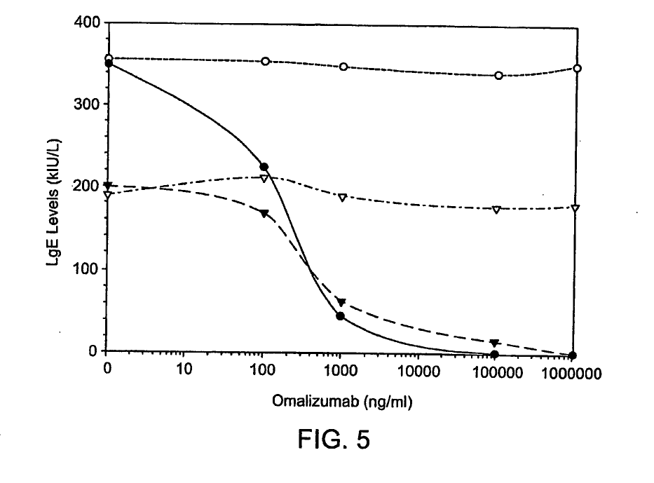

Once administered, maximal decreases in free circulating IgE have been reported when in vivo [IgG1-anti-IgE:IgE] molar ratios in blood of 10 to 20 are achieved (Casale et al, 1997). Because the efficacy of Omalizumab involves IgE-anti-IgE complex formation and Omalizumab interference of IgE binding onto FcεR1, the level of free (uncomplexed) IgE can be viewed as one indicator of the effectiveness of Omalizumab administration. During the development of Omalizumab, a free IgE enzyme immunoassay was developed using a human IgE receptor alpha chain-IgG chimera (Haak-Frendscho et al, 1993). This chimeric reagent when insolublized on plastic surfaces of microtiter plate wells bound “free” IgE that was not complexed with Omalizumab. While the human FcεR1α-IgG chimera appears to be a useful reagent for immunoassay measurement of free IgE, it is not available for use by clinical laboratories. As an alternative, a recombinant form of the extracellular domain of the alpha chain of the FcεR1 has been produced in a Baculovirus expression system that appears to be a useful alternative (Stedman et al, 2001). However, when purified FcεR1α is insolubilized on plastic surfaces, it appears to lose its reactivity for IgE, most likely due to steric hindrance. For this reason, we chose the reciprocal assay configuration of capturing IgE with a solid phase anti-IgE and then detecting bound free IgE with biotin labeled-FcεR1α. This assay design has its strengths and limitations. It is able to capture all IgE (whether free or Omalizumab-bound) which means that sera with high total serum IgE levels can require that multiple dilutions be analyzed to insure that the measurement falls in the working range of the assay. Since baseline IgE levels for candidate Omalizumab patients are typically 30 to 700 krU/L, this is not a serious concern because these levels are generally within the working range of both the free and total serum IgE IEMAs. However, analysis of sera from patients already receiving Omalizumab may have to be diluted since the measured total serum IgE does increase five to six fold due to the longer biological half-life of the complexes. The increases in level of total circulating IgE reported (Casales et al, 1997) and observed in this study following Xolair administration have been ascribed to the molar excess concentrations of anti-IgE that produce and retain small anti-IgE:IgE complexes in circulation. This increases the biological half-life of IgE in serum. One additional advantage of the IEMAs' design is that total serum IgE can be monitored even in a serum that contains Omalizumab since binding of the humanized IgG1 anti-IgE to IgE does not appear to interfere in the measurement of selected research and clinically-used total serum IgE immunoassays (FIG. 5) (Hamilton, 2006).

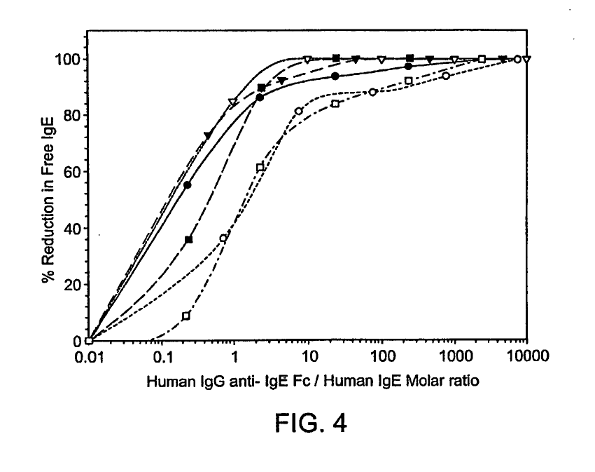

The free IgE level in serum when analyzed in tandem with the total IgE provides a snapshot into the status of IgE levels in patients on Omalizumab. Since not all patients on Omalizumab experience the same discernable symptom relief following Omalizumab treatment (Bousquet et al, 2004; Soler et al, 2001; Busse et al, 2001), the availability of a free IgE measurement may be a useful analytical measurement for verifying that a sufficient dose was administered and that the concentration of the Omalizumab is not simply the cause of an apparent lack of drug efficacy. In this study, two of the twelve patients on Omalizumab exhibited only a 50% reduction in free IgE despite an appropriate dosing based on the subject's weight and baseline total serum IgE level (Subjects 2 and 11, Table 1). Both of these patients had among the lowest baseline total serum IgE levels. In vitro studies with total IgE levels in this range demonstrate some heterogeneity in the amount of anti-IgE required to reduce free IgE levels (FIG. 4). Whether this is a result of the blocking of Omalizumab binding to IgE by naturally-occurring IgG anti-IgE antibodies (Lichtenstein et al, 1992; Ritter et al., 1991) is unclear and may need further investigation. Sustained increases in total serum IgE were observed even in these two subjects, suggesting that Omalizumab was complexing with their circulating IgE.

The present invention relates novels assays with acceptable performance characteristics that allow the monitoring of changes in the levels of circulating free blood fluid IgE that is not saturated with therapeutic agents which lower free blood fluid IgE levels, such as Omalizumab and the IgE peptide (Jansson, 2006). Analysis of the free IgE levels in paired pre and 1 to 3 month post therapeutic agent treatment sera may aid physicians in confirming adequate dosing and monitoring immunological changes resulting from adjustments in the dosing schedule of patients receiving the therapeutic agent who do not experience the anticipated clinical benefit.

### Capture Antibody

The capture antibody for binding total blood fluid IgE has two primary characteristics: 1) the antibody binds to the Fc region of IgE; and 2) the antibody does not bind to an antigenic determinant of the alpha (α) chain of the FcεR1α receptor. Preferably, the capture antibody has a relatively high affinity for IgE, in the range of 10⁸ to 10¹⁰ L/M (Hamilton et al., 1989). The capture antibody can be a polyclonal or monoclonal antibody with monoclonal being the preferred embodiment. Suitable monoclonal antibodies for the methods and assays of the present invention can be obtained from commercial sources, or alternatively, they can be developed using conventional hybridoma technology. Commercial sources of monoclonal capture antibodies include EMD Biosciences Corporation of La Jolla, Calif. and the Hybridoma Reagent Laboratory of Baltimore, Md. Both mouse IgM anti-human IgE Fc clone HP6061 and mouse IgG1 anti-human IgE Fc clone HP6029 are available from these commercial sources.

As mentioned, other hybridomas producing anti-IgE immunoglobulins can be developed using standard hybridoma technology. See, for example, Kohler & Milstein, Nature 256:495 (1975); Coligan et al., sections 2.5.1-2.6.7; and Harlow et al., ANTIBODIES: A LABORATORY MANUAL, page 726 (Cold Spring Harbor Pub. 1988), which are hereby incorporated by reference. Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., Purification of Immunoglobulin G (IgG), in METHODS IN MOLECULAR BIOLOGY, VOL. 10, pages 79-104 (Humana Press 1992). Methods of in vitro and in vivo multiplication of monoclonal antibodies is well-known to those skilled in the art. Multiplication in vitro may be carried out in suitable culture media such as Dulbecco's Modified Eagle Medium or RPMI 1640 medium, optionally replenished by a mammalian serum such as fetal calf serum or trace elements and growth-sustaining supplements such as normal mouse peritoneal exudate cells, spleen cells, bone marrow macrophages. Production in vitro provides relatively pure antibody preparations and allows scale-up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, e.g., syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Antibodies of the invention also may be derived from antibody fragments isolated from a combinatorial immunoglobulin library. See, for example, Barbas et al., METHODS: A COMPANION TO METHODS IN ENZYMOLOGY, VOL. 2, page 119 (1991); Winter et al., Ann. Rev. Immunol. 12: 433 (1994), which are hereby incorporated by reference. Cloning and expression vectors that are useful for producing a immunoglobulin phage library can be obtained, for example, from STRATAGENE Cloning Systems (La Jolla, Calif.).

In addition, antibodies of the present invention may be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994); Lonberg et al., Nature 368:856 (1994); and Taylor et al., Int. Immunol. 6:579 (1994), which are hereby incorporated by reference.

Antibody fragments of the present invention can be prepared by proteolytic hydrolysis of the antibody or by expression in E. Coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using papain produces two monovalent Fab fragments and an Fc fragment directly. These methods are described, for example, by Goldenberg, U.S. Pat. Nos. 4,036,945 and 4,331,647, and references contained therein. These patents are hereby incorporated in their entireties by reference. See also Nisonhoff et al., Arch. Biochem. Biophys. 89:230 (1960); Porter, Biochem. J. 73:119 (1959); Edelman et al., METHODS IN ENZYMOLOGY, VOL. 1, page 422 (Academic Press 1967); and Coligan et al. at sections 2.8.1-2.8.10 and 2.10.1-2.10.4.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be non-covalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde. See, e.g., Sandhu, supra. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow et al., METHODS: A COMPANION TO METHODS IN ENZYMOLOGY, VOL. 2, page 97 (1991); Bird et al., Science 242:423-426 (1988); Ladner et al., U.S. Pat. No. 4,946,778; Pack et al., Bio/Technology 11: 1271-77 (1993); and Sandhu, supra.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al., METHODS: A COMPANION TO METHODS IN ENZYMOLOGY, VOL. 2, page 106 (1991).

### FcεR1 (High Affinity IgE Receptor)

According to the present invention, an isolated, or biologically pure, FcεR1, is a molecule that has been removed from its natural milieu. As such, “isolated” and “biologically pure” do not necessarily reflect the extent to which the molecule has been purified. An isolated human FcεR1 of the present invention can be obtained from its natural source (e.g., from a human mast cell), can be produced using recombinant DNA technology or can be produced by chemical synthesis.

A FcεR1 of the present invention can be a full-length protein, a portion of a full-length protein or any homolog of such a protein. As used herein, a protein can be a polypeptide or a peptide. A FcεR1 of the present invention can comprise a complete high affinity Fcε receptor (i.e., alpha, beta and gamma chains of the FcεR), an alpha chain of the FcεR1 (also referred to herein as FcεR1α) or portions thereof. Preferably, a FcεR1 comprises at least a portion of a FcεR1 alpha chain that binds to IgE, i.e., that is capable of forming an immune complex with an IgE epsilon heavy chain constant region. Preferably, a FcεR1α of the present invention binds to IgE with an affinity of about K_{A} 10⁸, more preferably with an affinity of about K_{A} 10⁹ and even more preferably with an affinity of about K_{A} 10¹⁰ L/M.

An isolated FcεR1α of the present invention, including a homolog, can be identified in a straight-forward manner by the FcεR1α's ability to form an immune complex with IgE. Examples of FcεR1α receptor homologs include FcεR1α proteins in which amino acids have been deleted (e.g., a truncated version of the protein, such as a peptide), inserted, inverted, substituted and/or derivatized (e.g., by glycosylation, phosphorylation, acetylation, myristoylation, prenylation, palmitoylation, amidation and/or addition of glycerophosphatidyl inositol) such that the homolog includes at least one epitope capable of forming an immune complex with an IgE molecule.

FcεR1α homologs can be the result of natural allelic variation or natural mutation. FcεR1α homologs of the present invention can also be produced using techniques known in the art including, but not limited to, direct modifications to the protein or modifications to the gene encoding the protein using, for example, classic or recombinant DNA techniques to effect random or targeted mutagenesis.

According to the present invention, a human FcεR1 alpha. chain protein of the present invention is encoded by at least a portion of the nucleic acid sequence of the coding strand of a cDNA encoding a full-length FcεR1 alpha chain protein represented herein as SEQ ID NO 13 as set forth in U.S. Pat. No. 5,945,294. It is encoded by SEQ ID NO:12. Specifically, the amino acid sequence stretches from the 1st amino acid after the signal sequence through amino acid 172 (where amino acid 1 is the 1st amino acid after the signal sequence).

Isolated FcεR1α protein of the present invention can be produced by culturing a cell capable of expressing the protein under conditions effective to produce the protein, and recovering the protein. A preferred cell to culture is a recombinant cell that is capable of expressing the protein, the recombinant cell being produced by transforming a host cell with one or more nucleic acid molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained. Suitable and preferred nucleic acid molecules with which to transform a cell are as disclosed herein for suitable and preferred FcεR1α nucleic acid molecules per se.

Suitable host cells to transform include any cell that can be transformed with a nucleic acid molecule of the present invention. Host cells can be either untransformed cells or cells that are already transformed with at least one nucleic acid molecule. Host cells of the present invention either can be endogenously (i.e., naturally) capable of producing a FεR1α receptor protein of the present invention or can be capable of producing such proteins after being transformed with at least one nucleic acid molecule of the present invention. Host cells of the present invention can be any cell capable of producing at least one protein of the present invention, and include bacterial, fungal (including yeast), parasite (including protozoa and ectoparasite), insect, other animal and plant cells.

Preferably, a recombinant cell is transfected with a recombinant molecule of the present invention is a molecule that can include at least one of any nucleic acid molecule heretofore described operatively linked to at least one of any transcription control sequence capable of effectively regulating expression of the nucleic acid molecule(s) in the cell to be transformed, examples of which are disclosed herein.

A FcεR1α of the present invention can be contained in a formulation, herein referred to as a FcεR1α formulation. For example, a FcεR1α can be combined with a buffer in which the FcεR1α is solubilized, and/or a carrier. Suitable buffers and carriers are known to those skilled in the art. Examples of suitable buffers include any buffer in which a FcεR1α can function to selectively bind to IgE, such as, but not limited to, phosphate-buffered saline, water, saline, phosphate buffer, bicarbonate buffer, HEPES buffer (N-2-hydroxyethylpiperazine-N′-2-ethanesulfonic acid buffered saline), TES buffer (Tris-EDTA buffered saline), Tris buffer and TAE buffer (Tris-acetate-EDTA). Examples of carriers include, but are not limited to, polymeric matrices, toxoids, and serum albumins, such as bovine serum albumin. Carriers can be in mixed with FcεR1α or conjugated (i.e., attached) to FcεR1α in such a manner as to not substantially interfere with the ability of the FcεR1α to selectively bind to IgE.

The present invention also includes human FcεR1α mimetopes and use thereof to detect IgE. In accordance with the present invention, a “mimetope” refers to any compound that is able to mimic the ability of a FcεR1α to bind to IgE. A mimetope can be a peptide that has been modified to decrease its susceptibility to degradation but that still retains IgE-binding activity. Other examples of mimetopes include, but are not limited to, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of synthetic compounds for compounds capable of binding to IgE. A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modeling. The predicted mimetope structures can then be produced by, for example, chemical synthesis, recombinant DNA technology, or by isolating a mimetope from a natural source. Specific examples of FcεR1α mimetopes include anti-idiotypic antibodies, oligonucleotides produced using Selex technology, peptides identified by random screening of peptide libraries and proteins identified by phage display technology.

As used herein, the term “contacting” refers to combining or mixing, in this case a putative IgE-containing composition with a human FcεR1α. Formation of a complex between a FcεR1α and an IgE refers to the ability of the FcεR1α to selectively bind to IgE in order to form a stable complex that can be measured (i.e., detected). As used herein, the term selectively binds to an IgE refers to the ability of a FcεR1α of the present invention to preferentially bind to IgE, without being able to substantially bind to other antibody isotypes (e.g., IgG, IgA, IgM and IgD). Binding between a FcεR1α and an IgE is effected under physiological conditions suitable to form a complex; such conditions (e.g., appropriate concentrations, buffers, temperatures, reaction times) as well as methods to optimize such conditions are known to those skilled in the art, and examples are disclosed herein. Examples of complex formation conditions are also disclosed in, for example, in Sambrook et al., Molecular Cloning: A Laboratory Manual, Cold Spring Harbor Labs Press, 1989, the reference Sambrook et al., ibid., is incorporated by reference herein in its entirety.

As used herein, the term “measuring the amount of detection label” refers to determining if any complex is formed, i.e., assaying for the presence (i.e., existence) of a complex. If complexes are formed, the amount of complexes formed can determined. Complex formation, or selective binding, between FcεR1α and any IgE in the composition can be measured (i.e., detected, determined) using a variety of methods standard in the art (see, for example, Sambrook et al. ibid.), examples of which are disclosed herein.

A complex can be detected in a variety of ways including, but not limited to use of one or more of the following assays: an enzyme-linked immunoassay, a radioimmunoassay, a fluorescence immunoassay, a chemiluminescent assay, a lateral flow assay, an agglutination assay, a particulate-based assay (e.g., using particulates such as, but not limited to, magnetic particles or plastic polymers, such as latex or polystyrene beads), an immunoprecipitation assay, a BioCore assay (e.g., using colloidal gold), Bioplex assay (antibody coated microparticles in a flow cytometry platform) and an immunoblotting assay (e.g., a Western blot). Such assays are well known to those skilled in the art. Assays can be used to give qualitative or quantitative results depending on how they are used. Some assays, such as agglutination, particulate separation, and immunoprecipitation, can be observed visually (e.g., either by eye or by a machines, such as a densitometer or spectrophotometer) without the need for a detectable marker. In other assays, conjugation (i.e., attachment) of a detectable marker to the FcεR1α or to a reagent that selectively binds to the FcεR1α or to the IgE being detected (described in more detail below) aids in detecting complex formation. Examples of detectable markers include, but are not limited to, a radioactive label, a fluorescent label, a chemiluminescent label, a chromophoric label or a ligand. A ligand refers to a molecule that binds selectively to another molecule. Preferred detectable markers include, but are not limited to, fluorescein, a radioisotope, a phosphatase (e.g., alkaline phosphatase), biotin, avidin, a peroxidase (e.g., horseradish peroxidase) and biotin-related compounds or avidin-related compounds (e.g., streptavidin or ImmunoPure® NeutrAvidin). Preferably, biotin is conjugated to the FcεR1 alpha chain. Preferably, a carbohydrate group of the FcεR1α is conjugated with biotin.

In one embodiment, a complex is detected by contacting a putative IgE-containing composition with a FcεR1α that is conjugated to a detectable marker. A suitable detectable marker to conjugate to a FcεR1α includes, but is not limited to, a radioactive label, a fluorescent label, a chemiluminescent label or a chromophoric label. A detectable marker is conjugated to a FcεR1α or a reagent in such a manner as not to block the ability of the FcεR1α or reagent to bind to the IgE being detected. Preferably, a carbohydrate group of a FcεR1α is conjugated to biotin.

Immobilization techniques are known to those skilled in the art. Suitable substrate materials include, but are not limited to, plastic, glass, gel, celluloid, paper, PVDF (poly-vinylidene-fluoride), nylon, nitrocellulose, and particulate materials such as latex, polystyrene, nylon, nitrocellulose, agarose and magnetic resin. Suitable shapes for substrate material include, but are not limited to, a well (e.g., microtiter dish well), a plate, a dipstick, a bead, a lateral flow apparatus, a membrane, a filter, a tube, a dish, a celluloid-type matrix, a magnetic particle, and other particulates. A particularly preferred substrate comprises an ELISA plate, a dipstick, a radioimmunoassay plate, agarose beads, plastic beads, latex beads, glass microbeads, immunoblot membranes and immunoblot papers. In one embodiment, a substrate, such as a particulate, can include a detectable marker.

### Methods of the Invention

The present invention includes to a method of quantifying the free blood fluid IgE level in a patient comprising the steps of a) contacting the patient's blood fluid with an IgE directed antibody immobilized on a substrate under conditions suitable for formation of a IgE:capture antibody complex; b) contacting the substrate of step (a) with a detection labeled FcεR1α under conditions suitable for formation of an IgE: FcεR1α complex; and c) determining the quantity of free blood fluid IgE by measuring the amount of detection label in comparison to known quantity level standards for IgE.

In a preferred embodiment, capture antibody is a monoclonal antibody, and in particular,

wherein the monoclonal antibody binds to the Fc portion of the IgE and does not bind to an antigenic determinant of the α-chain of the FcεR1α receptor. In particular embodiments, the capture antibody is the monoclonal murine IgM anti-human IgE Fc clone HP6061 or the monoclonal murine IgG1 anti-human IgE Fc clone 1326029. In still other embodiments, the patient's blood fluid is comprised of serum or plasma.

Further other embodiments include the FcεR1α comprising at least a portion of a human Feε R alpha chain that binds IgE, particularly from the FcεR1α receptor comprising the amino acid sequence of SEQ ID No. 1 or the nucleic acid sequence of SEQ ID No.1 encoding the FcεR1α receptor.

The FcεR1α can be conjugated to a detection label selected from the group consisting of a radioactive label, a fluorescent label, a chemiluminescent label, a chromophoric label and a ligand or more particularly, a detection label selected from the group consisting of fluorescein, a radioisotope, a phosphatase, biotin, biotin-related compounds, avidin, avidin-related compounds and a peroxidase.

In another preferred embodiment of the present invention, the substrate comprises a material selected from the group consisting of plastic, glass, gel, celluloid, paper, particulate material and latex, polystyrene, nylon, nitrocellulose, agarose and magnetic resin. Furthermore, the substrate can comprise a shape selected from the group consisting of a well, a plate, a dipstick, a bead, a lateral flow apparatus, a membrane, a filter, a tube, a dish, a celluloid-type matrix and a magnetic particle. Such substrates can be made as an ELISA plate, a dipstick, a radioimmunoassay plate, agarose beads, plastic beads, latex beads, immunoblot membranes and immunoblot papers.

The present invention also relates to quantitative determination of free blood fluid IgE levels performing assays selected from the group consisting of enzyme-linked immunoassays, radioimmunoassays, immunoprecipitations, fluorescence immunoassays, chemiluminescent assay, immunoblot assays, lateral flow assays, agglutination assays and particulate-based assays.

Another embodiment of the present invention includes a method of determining the efficacy of a therapeutic agent that lowers a patient's free blood fluid IgE level by quantifying the patient's free blood fluid IgE levels before and after administration of the therapeutic agent comprising the steps of: a) contacting the patient's blood fluid with an IgE directed antibody immobilized on a substrate under conditions suitable for formation of a IgE:capture antibody complex; b) contacting the substrate of step (a) with a detection labeled FcεR1α under conditions suitable for formation of an IgE: FcεR1α complex;

c) determining the quantity of free blood IgE by measuring the amount of detection label in comparison to known quantity level standards for IgE; d) comparing the patient's free blood fluid IgE levels before and after administration of the therapeutic agent. In a preferred embodiment, the patient's blood fluid is serum, capture antibody binds to the Fc portion of the IgE and does not bind to an antigenic determinant of the α-chain of the FcεR1 and the therapeutic agent is omalizumab.

### Assay Kits

The present invention also includes kits to detect IgE based on each of the disclosed detection methods. One embodiment is a kit to detect IgE comprising a human FcεR1α molecule and a means for detecting an IgE. Suitable and preferred FCε receptors are disclosed herein. Suitable means of detection include compounds disclosed herein that bind to either the FcεR1α or to an IgE. A preferred kit of the present invention further comprises a detection means disclosed herein, an antibody capable of selectively binding to an IgE disclosed herein and/or a compound capable of binding to a detectable marker conjugated to a FcεR1α (e.g., avidin and streptavidin when the detectable marker is biotin).

All publications and patent applications cited in this specification are herein incorporated by reference as if each individual publication or patent application were specifically and individually indicated to be incorporated by reference.

### EXEMPLIFICATION

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

### Materials and Methods:

### Reagents:

Pharmaceutical grade Omalizumab was purchased from Genentech (South San Francisco, Calif.) and reconstituted as recommended in the package insert. The 147 mg/ml stock was diluted further with phosphate buffered saline containing 1% bovine serum albumin (PBS-BSA) to prepare Omalizumab (anti-IgE) concentrations from 100 ng/ml to 1 mg/ml. These Omalizumab preparations were incubated overnight with human serum containing known quantities of IgE to prepare complexes at [IgG-anti-IgE:IgE] molar ratios from 0.22 to 9500.

The anti-human IgE monoclonal antibodies used as capture and detection reagents were produced from clones HP6061 (mouse IgM anti-human IgE Fc) and HP6029 (mouse IgG1 anti-human IgE Fc) (Reimer, 1986), respectively. Both clones were prepared using PS-IgE myeloma as the immunogen and purchased as chromatographically purified antibody at 2 mg/ml in PBS from EMD Biosciences Corporation (La Jolla, Calif.). Both purified monoclonal antibodies were biotinylated using the biotin-hydroxysuccinimide ester method (EMD Biosciences, La Jolla Calif.) and stored frozen at 1 mg/ml in PBS-BSA.

A recombinant form of the extracellular domain of the alpha chain of the human high affinity IgE receptor (FcεR1α) was purchased from Heska Corporation (Fort Collins, Colo.). It was produced in a Baculovirus expression system as previously described (Stedman et al. 2001) and was subsequently purified by Q-Sepharose anion exchange chromatography and gel filtration. Purified FcεR10α was biotinylated with biotin hydrazide using the periodate oxidation method.

### Human Sera:

The human sera that were used to construct the [IgG-anti-IgE:IgE] complexes were obtained from healthy adults with total serum IgE levels ranging from <2 to 100,000 kIU/L. Moreover, serum from 33 adults pre-Omalizumab treatment were collected for correlation studies to compare the free and total serum IgE IEMA results with total serum IgE measurements obtained in the ImmunoCAP 250 (Pharmacia, Kalamazoo, Mich.) (Hamilton and Adkinson, 2004). In the prospective Omalizumab treatment study, serum was collected from twelve patients with allergic asthma before Omalizumab treatment (baseline) and at 1 and 3 months during Omalizumab treatment. All patients received 0.016 mg of Omalizumab per kg of body weight per IU/ml of baseline total serum IgE for the management of their allergic asthma (Table 1). All sera were used to examine various aspects of the analytical performance of the free and total IgE IEMAs in the absence and presence of Omalizuamb. Sera were collected in accordance with procedures approved by the Johns Hopkins Bayview Institutional Review Board.

### Free Human IgE Immunoenzymetric Assay.

The concentration of (non-Omalizumab bound) free IgE in human serum was measured using a solid phase IEMA in which IgE was captured from serum with anti-human IgE and detected with labeled-FcεR1α. Monoclonal murine IgM anti-human IgE Fc (Clone HP6061P) was adsorbed onto sterile flat-bottom polystyrene 96 well Bacti plates (Nalge-Nunc International, Rochester, N.Y.) by pipetting 0.1 ml per well of a 10 microgram per ml solution in PBS (pH 7.4, 16-18 hrs at 2°-8° C.). The plates were washed once with PBS containing 0.05% Tween 20 and 0.01% thimerosal (PBS-Tween) and blocked with 300 microliters per well of blocking buffer (PBS-1% BSA, 0.01% thimerosal). Following 5 buffer washes, 50 microliters of blocking buffer were distributed in all wells. Then, 50 microliters of each total IgE reference serum calibrators [11 two-fold dilutions of the 2000 kIU/L calibrator from 1000 to 1 kIU/L and total serum IgE high, medium and low control sera (Pharmacia, Kalamazoo, Mich.) and test sera were pipetted into their respective wells. If serum had to be diluted to insure the IgE level in the specimen was within the working range of the assay, a serum from a non-atopic blood donor (IgE level <2 kIU/L) was used as the diluent. Plates were incubated 2 hours at 37° C. and then without any buffer wash, biotinylated-FcεR1α was pipetted into all wells (3 micrograms per ml, 0.05 ml/well). The serum—biotin-FcεR1α mixture was incubated 1 hour at 37° C. Following 5 PBS-Tween washes, avidin-horseradish peroxidase (Sigma Chemical Company, St. Louis, Mo.) was pipetted into each well (1 microgram per ml, 0.1 ml/well) and the plate was incubated 1 hour at 37° C. Following this final incubation, the plates were washed 5 times with PBS-Tween and ABTS substrate, containing 1 microliter of H₂0₂ per ml of ABTS, was pipetted into each well (0.1 ml/well). The enzymatic reaction was stopped with 0.1 ml of 1M sodium azide per well when the top point of the reference curve reached an optical density of 1.5 to 2.0. Each plate was read in an ELISA plate reader (MR4000, Dynatech Technologies, Maclean, Va.) at 405 nm.

Optical density of the IgE calibrators was plotted versus IgE concentration and test serum optical density values were interpolated into free IgE concentrations in kIU/L from the calibration curve. Acceptance of each assay was based on the low (16-28 kIU/L), medium (69-117 kIU/L) and high (300-500) total serum IgE controls being in their target range and the intra-assay CV for each specimen being <15%. The interpolated IgE concentrations in the free IgE IEMA were deemed to be “free” IgE levels because they were able to bind labeled-FcεR1α (Casale T B et al, 1997). Intra-assay variation of the free IgE IEMA was assessed by precision profile analysis in which 180 duplicate were performed in 5 different assays with sera containing IgE levels across the working range of the assay (10 to 1000 kIU/L). Inter-assay variation was assessed by analyzing sera containing IgE levels across the working range of the assay in multiple assays performed on separate days. Parallelism (linearity) was assessed by computing the inter-dilutional coefficients of variation of specimens analyzed in the same assay at multiple dilutions.

### Total Serum IgE Immunoenzymetric Assay.

Total serum IgE levels in the test sera were measured using a previously reported monoclonal antibody-based IEMA (Hamilton and Adkinson, 1992) and the ImmunoCAP 250 (Pharmacia, Kalamazoo, Mich.). The total serum IgE ImmunoCAP is an FDA-cleared solid phase immunometric assay in which anti-IgE that is attached to a CAP matrix binds IgE from serum. Following a wash step, labeled anti-human IgE is then added to quantitatively detect bound IgE. The total serum IgE IEMA involved the identical procedure described for the free serum IgE IEMA, except that the plate was washed between the serum incubation and biotin-anti-human IgE addition, and bound IgE was detected with biotinylated monoclonal murine IgG1 anti-human IgE Fc (clone HP6029) at 1 microgram per ml (0.1 ml/well). The monoclonal antibody pair (clones HP6061 for capture and HP6029-biotin for detection) are reported to bind to different determinants on the Fc region of the human epsilon heavy chain (Hamilton and Adkinson, 1992). Neither antibody impedes the binding of IgE to the FcεR1α on basophils and both activate histamine release in sensitized basophils. The total serum IgE IEMA in this study was calibrated using commercially-available human IgE standards (Pharmacia, Kalamazoo, Mich.) that were traceable to the World Health Organization IgE Standard. Since baseline IgE levels will be measured in clinical laboratories using one of several clinical assays such as the ImmunoCAP, we chose to compare the total serum IEMA results obtained in this study to those generated in the ImmunoCAP-250 with the same sera.

Omalizumab (anti-IgE):IgE Complex Preparation in Varying Molar Ratios.

The performance of the free IgE IEMA was evaluated using a matrix of [human IgG1 anti-human IgE to human IgE] complexes with molar ratios spanning the therapeutically reported range (1 to 20) (Casale et al, 1997). In the present study, human sera containing IgE levels from 111.4 to 480 kIU/L were mixed with equal volumes of buffer (0 control) or one of 5 ten-fold concentrations of Omalizumab (100 ng/ml to 1 mg/ml). The final IgE concentration in the absence of Omalizumab (buffer control condition) ranged from 55.7 to 240 kIU/L. Omalizumab to human IgE molar ratios ranged from 0.22 to 9500, adjusting for the different molecular weights of IgG and IgE. The [sera:buffer] or [serum:Omalizumab] mixtures were incubated at least 2 hours at room temperature to allow complex formation prior to their analysis in the free and total IgE IEMAs.

### Data and Statistical Analyses

All IEMA data were computed using a spline curve-fitting algorithm in a Johns Hopkins University DACI Laboratory Immunoassay Data Processing program. Precision profile analysis involving assessment of precision of the dose as a function of the measured dose across the working range of the assay was performed as previously described (Ekins, 1991). Coefficients of variation (CV) of replicate data and linear regression analysis to assess relationships between the free and total serum IgE levels of the various assays were performed using commercial software (Microsoft Excel and SPSS).

### EXAMPLES

### Example I

### Free IgE IEMA

In preliminary experiments, various combinations and concentrations of the available human IgE specific immunochemical reagents were evaluated for their utility in the free IgE IEMA. Monoclonal anti-human IgE (clones HP6029 and HP6061) and FcεR1α were used in different combinations either as a purified reagent at 2.5, 5, 10, 20 micrograms per ml on the plate to capture IgE or as a biotinylated human IgE detection reagent at 0.5, 1, 2 and 4 micrograms per ml. To mimic a previously reported free IgE assay configuration (Casale et al, 1997), FcεR1α was initially insolubilized on plastic plates and used in combination with either clone of biotinylated anti-human IgE monoclonal detection antibody. However, purified FcεR1α displayed poor IgE reactivity when adsorbed directly onto a plastic surface. The optimal free IgE IEMA configuration was anti-human IgE (clone HP6061) adsorbed on the microtiter plate at 10 micrograms per ml to capture IgE from serum and biotinylated FcεR1α at 3 microgram per ml to detect bound IgE that was free of Omalizamub binding. Elimination of the wash step between the addition of serum and the biotinylated-FcεR1α minimized in vitro dissociation of the pre-formed [IgG anti-IgE:IgE] complexes and maximized analytical sensitivity of the free IgE IEMA (data not shown).

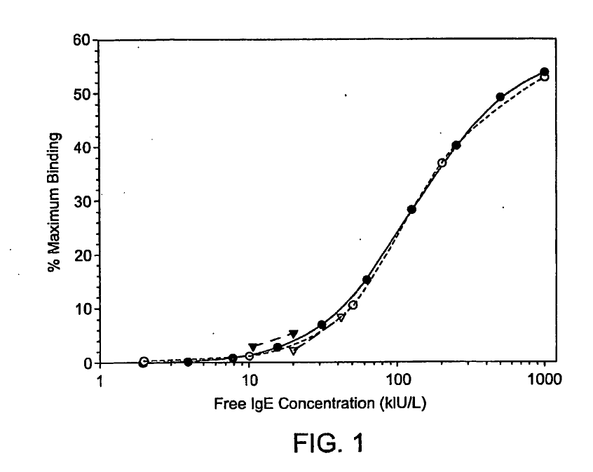

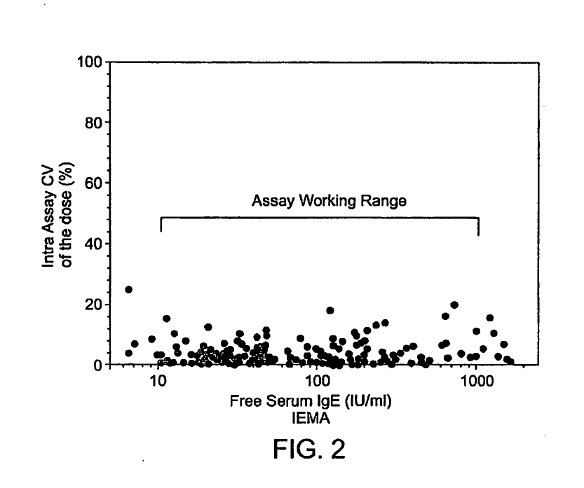

The assay in its optimal configuration displayed a working range of 10 to 1000 kIU/L in the absence of exogenously administered Omalizumab (FIG. 1). Precision profile analysis demonstrated acceptable precision with intra-assay coefficients of variation <15% over the working range of the assay (FIG. 2). Reproducibility was demonstrated with inter-assay coefficients of variation <18%. Parallelism (linearity) was shown with inter-dilutional CVs <20% (illustrated in FIG. 1).

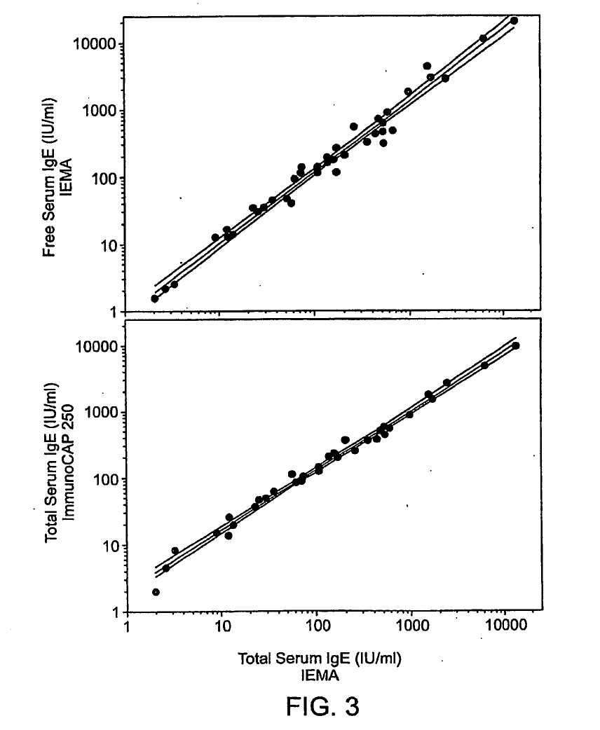

Regression analysis demonstrated that the free serum IgE levels in 33 pre-Omalizumab treatment sera were statistically correlated with the total serum IgE in the same sera as measured in the IEMA and ImmunoCAP 250 (FIG. 3). FIG. 4 displays the effect of increasing concentrations of Omalizumab on the percentage reduction in free serum IgE. The concentration of free IgE detected in the IEMA decreased as the anti-human IgE concentration increased from 100 ng/ml to 1 mg/ml. Free IgE decreases >90% occurred at in vitro-formed [IgG1-anti-IgE:IgE] molar ratios of 2 to 20. Total serum IgE IEMA results in the absence and presence of varying concentrations of Omalizumab were equivalent, indicating that Omalizumab levels as high as 1 mg/ml did not detectably interfere in the total serum IgE IEMA (FIG. 5).

### Example II

### Free and Total IgE Measurements in Patients on Omalizumab

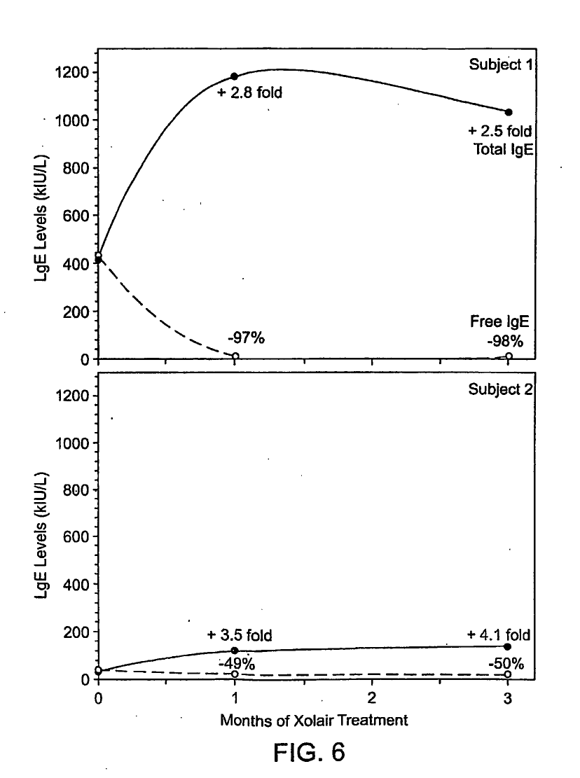

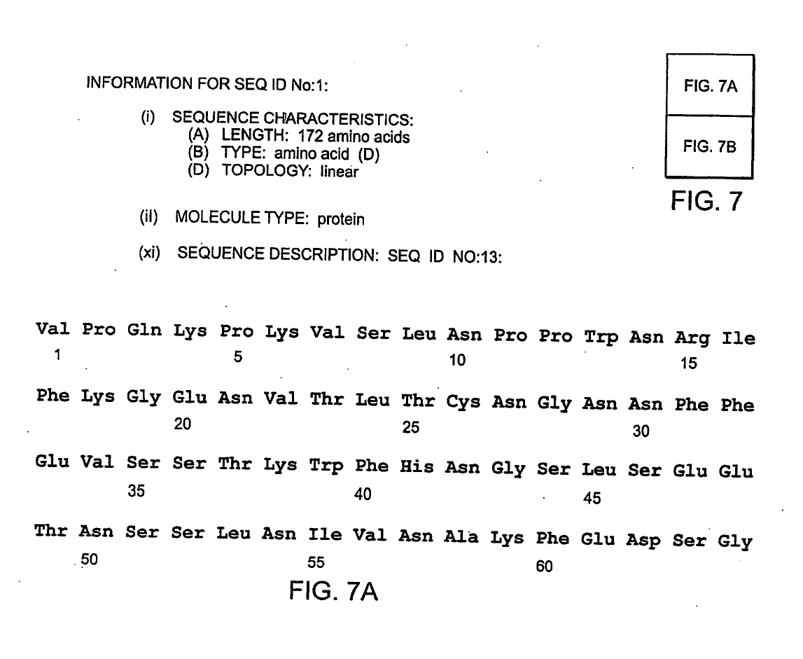

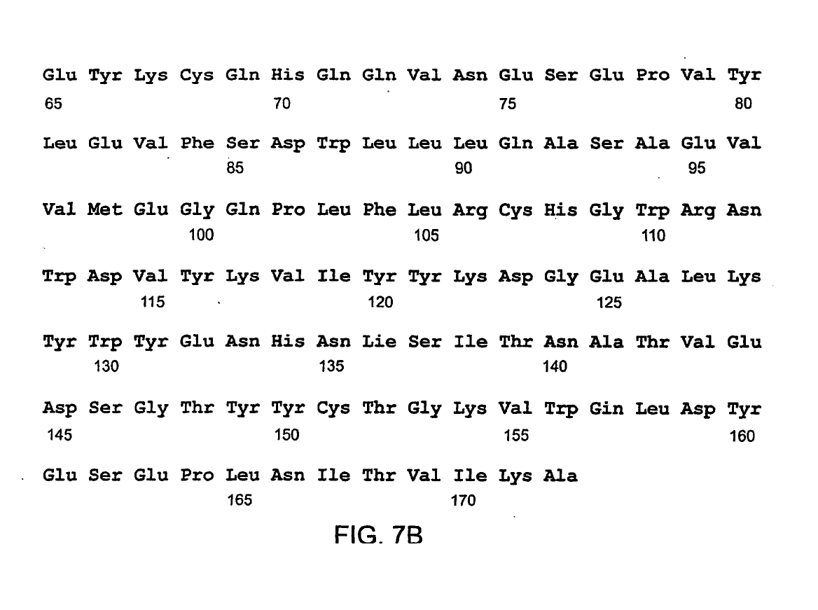

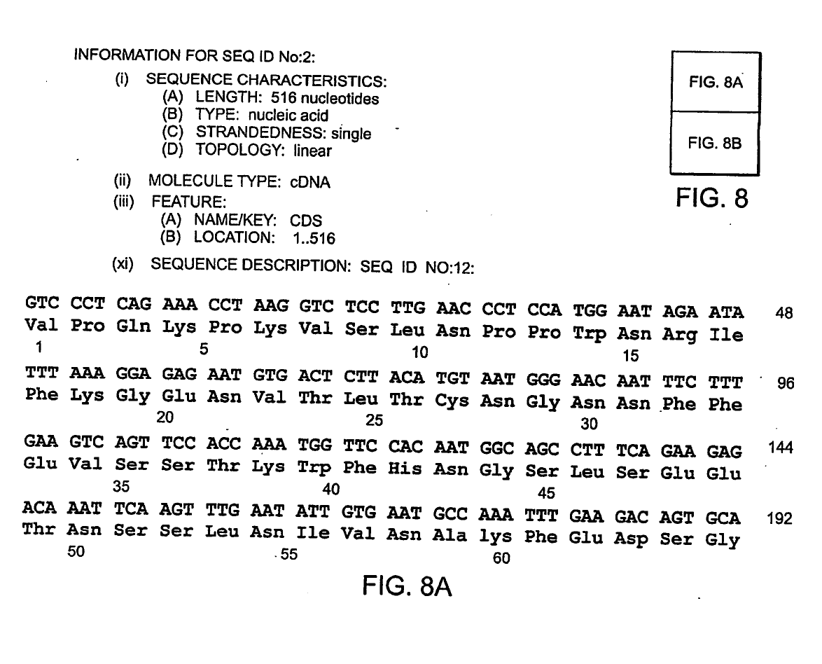

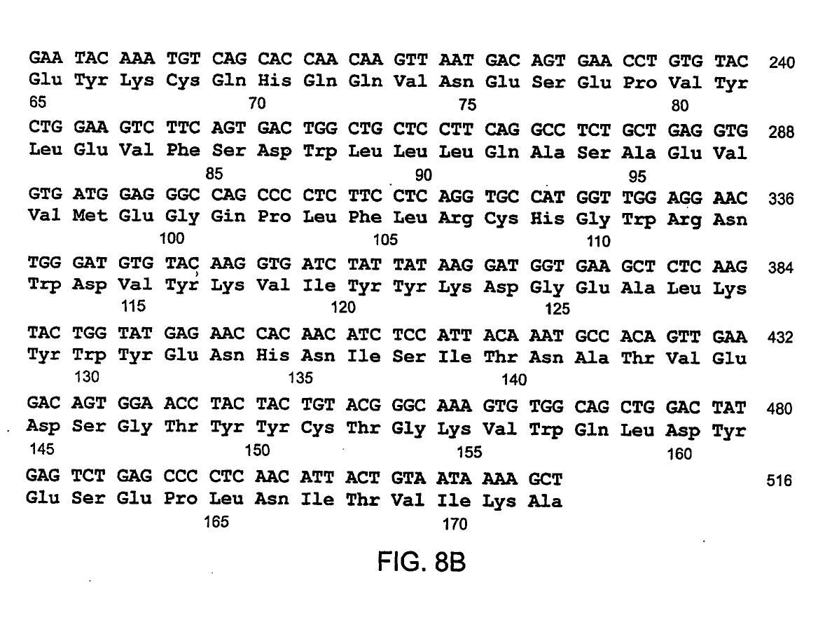

Table 1 presents the demographics and free and total IgE serological results at baseline and 1 and 3 months following continuous Omalizumab therapy in 12 subjects with asthma. The patient group included 6 females and 6 males with ages ranging from 41 to 75. Baseline (pre-Omalizumab treatment) total serum IgE and free IgE levels as measured by IEMA agreed well with each other. The total serum IgE increased from 1.5 to 8.6 times baseline levels depending on the individual and the time interval after initiation of Omalizmab treatment (Table 1). In contrast, levels of free IgE (unbound with Omalizumab) decreased in all patients, but to varying degrees. FIG. 6 displays the total and free IgE levels at the baseline, 1 and 3 month time points in two patients (Subjects 1 and 2) who displayed among the highest and lowest decreases in free IgE following Omalizumab administration. Subject 1 achieved a 97 and 98% reduction in free IgE levels at 1 and 3 months with a 2.5-2.8 fold increase in total IgE levels over the same time period. In contrast, subject 2 achieved only a 49% (1 month) and 50% (3 month) reduction in the free IgE level from a baseline free IgE of 41 kIU/L. Since minimal changes in free IgE were observed between the 1 and 3 month serum specimen, a 1 month post-treatment serum specimens was considered satisfactory for monitoring changes in free IgE levels in patients receiving Omalizumab treatment.

All publications and patent documents cited in this application are incorporated by reference in their entirety for all purposes to the same extent as if each individual publication or patent document were so individually denoted. These documents include U.S. Pat. Nos. 5,945,294; 6,309,832; and 6,682,894 and U.S. Provisional Patent Application 60/646,893. By their citation of various references in this document, Applicants do not admit any particular reference is “prior art” to their invention.

The following documents also are referred to herein by reference to author name or otherwise and are also incorporated herein by reference.
Bousquet, J., Wenzel, S., Holgate, S., Lumry, W., Freeman, P., Fox, H.2004 Predicting response to Omalizmab, an anti-IgE antibody, in patients with allergic asthma. Chest 125, 1378.
Busse, W., Corren, J., Lanier, B. Q., McAlary, M., Fowler-Taylor, A., Chioppa, G. D. 2001. Qmalizumab, anti-IgE recombinant humanized monoclonal antibody, for the treatment of severe allergic asthma. J. Allergy Clin Immunol 108:184-90.
Casale, T. B., Bernstein, I. L., Bussse, W. W., LaForce, C. F., Tinkelman, D. G., Stoltz, R. R., Dockhorn, R. J., 1997. Use of an anti-IgE humanized monoclonal antibody in ragweed induced allergic rhinitis. J. Allergy Clin. Immunol. 100, 110.
Chang, T. W., Davis, F. M., Sun, N. C., Sun, C. R. Y., MacGlashan, D. W., Hamilton, R. G. 1990. Monoclonal antibodies specific for human IgE producing B-cells: a potential therapeutic for IgE mediated allergic diseases. Biotechnology 9, 122.
Davis, L. A., 2004. Omalizumab: a novel therapy for allergic asthma. Ann. Pharmacotherapy 38, 1236.
Ekins, R. 1991. Competitive, non-competitive and multi-analyte microspot immunoassays. In. Butler, J. E., ed Immunochemistry of solid phase immunoassay, Boca Raton Fla.: CRC Press, p 105.
Haak-Frendscho. M., Ridgeway, J., Shields, R., Robbins, K., Gorman, C., Jardieu, P. 1993. Human IgE receptor alpha-chain IgG chimera blocks passive cutaneous anaphylaxis reaction in vivo. J. Immunol. 151, 351.
Hamilton, R. G., Sun, L., Beall, P. T. 1989. Production and quality control of monoclonal antibody reagents and drugs. In Moulds J. M. and Masouredis S. P. eds, “Monoclonal antibodies”. American Association of Blood Banks, Arlington Va., pg 83.—ISBN No 0-9153555-70-1.
Hamilton, R. G., Adkinson, N. F. Jr. 1992. Measurement of total serum immunoglobulin E and allergen-specific immunoglobulin E antibody. In. Rose, N., de Macario, E. C., Fahey, J. L., Friedman, H., Penn, G., Manual of Clinical Immunology, 5^{th} ed, Washington DC, American Society for Microbiology p 689.
Hamilton, R. G., Adkinson, N. F., Jr. 2004. In vitro assays for IgE mediated sensitivities. (Review) J. Allergy Clin. Immunol. 114, 213.
Hamilton, R. G., 2006 Accuracy of Food and Drug Administration-cleared IgE antibody assays in the presence of anti-IgE (Omalizumab). J Allergy Clin Immunol. 117:759.
Holgate, S. T., Chuchalin, A. G., Hebert, J., Lotvall. J., Persson. G. B., Chung, K. F., Bousquet, J. 2004. Efficacy and safety of a recombinant anti-immunoglobulin E antibody (Omalizumab) in severe allergic asthma. Clin. Exp. Allergy 34, 632.
Jansson, A., Fuentes A., Landstrom E., Person S., Xu B. 2006. IgE immunotherapy efficiently induces neutralizing anti-human IgE antibodies in primates. J Allergy Clin Immunol. 117:S279 (Abstract # 1079).
Lichtenstein, L. M., Kagey Sobotka, A., White, J. M., Hamilton, R. G. 1992. Anti-human IgG causes basophil histamine release by acting on IgG-IgE complexes bound to IgE receptors. J. Immunol. 148, 3929.
Lin, H, Boesel, K. M., Griffith, D. T., Prussin, C., Foster, B., Romero, F. A., Townley, R., Casale, T. B. 2004. Omalizumab rapidly decreases nasal allergic response and FcaRI on basophils. J. Allergy Clin. Immunol. 113, 297.
Naclerio, R., 1991. Allergic rhinitis [review]. N. Engl. J. Med. 325. 860.
Norman, P. S., 2004. Immunotherapy: 1999-2004. J. Allergy Clin. Immunol. 113, 1013.
Reimer, C. B. 1986. Five hybridomas secreting monoclonal antibodies against human IgE. Monoclonal Antibody News 4, 2.
Ritter, C., Battig, M., Kraemer, R., Stadler, B. M. 1991. IgE hidden in immune complexes with anti-IgE autoantibodies in children with asthma. J. Allergy Clin. Immunol. 88, 793.
Saini, S. S., MacGlashan, D. W., Sterbinsky, S. A., Togias, A., Adelman, D. C., Lichtenstein, L. M., Bochner, B. S. 1999. Down-regulation of human basophil IgE and Fc epsilon R1 alpha surface densities and mediator release by anti-IgE infusions is reversible in vitro and in vivo. J. Immunol. 162, 5624.
Soler, M., Matz, J., Townley, R., Buhl, R., O′Brien, J., Fox, H. 2001. The anti-IgE antibody Omalizumab reduces exacerbations and steroid requirement in severe allergic asthma. Eur. Respir. J. 18, 254.
Stedman, K, Lee, K., Hunter, S., Rivoire, B., McCall, C., Wassom, D. 2001. Measurement of canine IgE using the alpha chain of the human high affinity IgE receptor. Veterin. Immunol. Immunopath. 78, 349.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

We claim:

1. A method of quantifying the free blood fluid IgE level in a patient comprising the steps of:
a) contacting the patient's blood fluid with an IgE directed antibody immobilized on a substrate under conditions suitable for formation of a IgE:capture antibody complex;
b) contacting the substrate of step (a) with a detection labeled FcεR1α receptor under conditions suitable for formation of an IgE: FcεR1α complex; and
c) determining the quantity of free blood fluid IgE by measuring the amount of detection label in comparison to known quantity level standards for IgE.

2. The method of claim 1, wherein the capture antibody is a monoclonal antibody.

3. The method of claim 2, wherein the monoclonal antibody binds to the Fc portion of the IgE and does not bind to an antigenic determinant of the α-chain of the FcεR1.

4. The method of claim 2, wherein the monoclonal antibody is an IgG antibody

5. The method of claim 3, wherein the monoclonal antibody is an IgM antibody.

6. The method of claim 5, wherein the IgM antibody is a monomer.

7. The method of claim 1, wherein the capture antibody is the monoclonal murine IgM anti-human IgE Fc clone HP6061.

8. The method of claim 1, wherein the capture antibody is the monoclonal murine IgG1 anti-human IgE Fc clone H6029.

9. The method of claim 1, wherein the patient's blood fluid is comprised of serum or plasma.

10. The method of claim 1, wherein the FcεR1α comprises at least a portion of a human FcεR1 alpha chain that binds IgE.

11. The method of claim 1, wherein the FcεR1α comprises an amino acid sequence of SEQ ID No. 1.

12. The method of claim 1, wherein the FcεR1α is encoded by the nucleic acid sequence of SEQ ID No.1.

13. The method of claim 1, wherein said FcεR1α is conjugated to a detection label selected from the group consisting of a radioactive label, a fluorescent label, a chemiluminescent label, a chromophoric label and a ligand.

14. The method of claim 1, wherein the FcεR1α is conjugated to a detection label selected from the group consisting of fluorescein, a radioisotope, a phosphatase, biotin, biotin-related compounds, avidin, avidin-related compounds and a peroxidase.

15. The method of claim 1, wherein a carbohydrate group of said FcεR1α is conjugated to biotin.

16. The method of claim 1, wherein said substrate comprises a material selected from the group consisting of plastic, glass, gel, celluloid, paper and particulate material.

17. The method of claim 1, wherein said substrate material is selected from the group consisting of latex, polystyrene, nylon, nitrocellulose, agarose and magnetic resin.

18. The method of claim 1, wherein said substrate comprises a shape selected from the group consisting of a well, a plate, a dipstick, a bead, a lateral flow apparatus, a membrane, a filter, a tube, a dish, a celluloid-type matrix and a magnetic particle.

19. The method of claim 1, wherein said substrate comprises an ELISA plate, a dipstick, a radioimmunoassay plate, agarose beads, plastic beads, glass beads, latex beads, immunoblot membranes and immunoblot papers.

20. The method of claim 1, wherein said step of detecting comprises performing assays selected from the group consisting of enzyme-linked immunoassays, radioimmunoassays, immunoprecipitations, fluorescence immunoassays, chemiluminescent assay, immunoblot assays, lateral flow assays, agglutination assays and particulate-based assays.

21. A method of determining the efficacy of a therapeutic agent that lowers a patient's free blood fluid IgE level by quantifying the patient's free blood fluid IgE levels before and after administration of the therapeutic agent comprising the steps of:
a) contacting the patient's blood fluid with an IgE directed antibody immobilized on a substrate under conditions suitable for formation of a IgE:capture antibody complex;
b) contacting the substrate of step (a) with a detection labeled FcεR1α under conditions suitable for formation of an IgE: FcεR1α complex;
c) determining the quantity of free blood IgE by measuring the amount of detection label in comparison to known quantity level standards for IgE;
d) comparing the patient's free blood fluid IgE levels before and after administration of the therapeutic agent.

22. The method of claim 21, wherein the capture antibody is the monoclonal murine IgM anti-human IgE Fc clone HP6061.

23. The method of claim 21, wherein the capture antibody is the monoclonal murine IgG1 anti-human IgE Fc clone HP6029.

24. The method of claim 21 wherein, the capture antibody binds to the Fc portion of the IgE and does not bind to an antigenic determinant that is bound by the α-chain of the FcεR1.

25. The method of claim 21, wherein the patients blood is comprised of serum or plasma.

26. The method of claim 21, wherein the therapeutic agent is omalizumab.

27. The method of claim 21, wherein the therapeutic agent is a vaccine.

28. A assay kit for quantifying the free blood fluid IgE level in a patient comprising a IgE capture antibody and a detection labeled FcεR1α.

29. The assay kit of claim 28, wherein the FcεR1α comprises an amino acid sequence of SEQ ID No. 1.

30. The assay kit of claim 28, wherein the FcεR1α is encoded by the nucleic acid sequence of SEQ ID No.1.

31. The kit of claim 28, wherein said FcεR1α is conjugated to a detection label selected from the group consisting of a radioactive label, a fluorescent label, a chemiluminescent label, a chromophoric label and a ligand.

32. The assay kit of claim 28, wherein said FcεR1α is conjugated to a detection label selected from the group consisting of fluorescein, a radioisotope, a phosphatase, biotin, a biotin-related compound, avidin, an avidin-related compound and a peroxidase.

33. The assay kit of claim 28, wherein a carbohydrate group of said FcεR1α is conjugated to biotin.

34. The assay kit of claim 28, wherein the capture antibody binds to the Fc portion of the IgE and does not bind to an antigenic determinant of the α-chain of the FcεR1.

35. The assay kit of claim 28, wherein the capture antibody is the monoclonal murine IgM anti-human IgE Fc clone HP6061.

36. The assay kit of claim 28, wherein the capture antibody is the monoclonal murine IgG1 anti-human IgE Fc clone HP6029.

37. The kit of claim 28, wherein said capture antibody is immobilized on a substrate.

38. The kit of claim 37, wherein said substrate comprises a material selected from the group consisting of plastic, glass, gel, celluloid, paper, magnetic resin, polyvinylidene-fluoride, nylon, nitrocellulose and particulate material.

39. The kit of claim 38, wherein said substrate material is selected from the group consisting of latex, polystyrene, nylon, nitrocellulose, agarose and magnetic resin.

40. The kit of claim 28, wherein said substrate comprises a shape selected from the group consisting of a well, a plate, a dipstick, a bead, a lateral flow apparatus, a membrane, a filter, a tube, a dish, a celluloid-type matrix and a magnetic particle.

41. The kit of claim 28, wherein said substrate comprises an ELISA plate, a dipstick, a radioimmunoassay plate, agarose beads, plastic beads, latex beads, immunoblot membranes and immunoblot papers.


SEQUENCE LISTING (2 sequences)

SEQ ID NO: 1 - protein, 172 aa, Homo sapiens
VPQKPKVSLNPPWNRIFKGENVTLTCNGNNFFEVSSTKWFHNGSLSEETNSSLNIVNAKFEDSAEYKCQHQQVNDSEPVYLEVFSDWLLLQASAEVVMEGQPLFLRCHGWRNWDVYKVIYYKDGEALKYWYENHNISITNATVEDSGTYYCTGKVWQLDYESEPLNITVIKA

SEQ ID NO: 2 - DNA, 516 bp, Homo sapiens
  CDS (1)..(516)
gtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaaatttgaagacagtgcagaatacaaatgtcagcaccaacaagttaatgacagtgaacctgtgtacctggaagtcttcagtgactggctgctccttcaggcctctgctgaggtggtgatggagggccagcccctcttcctcaggtgccatggttggaggaactgggatgtgtacaaggtgatctattataaggatggtgaagctctcaagtactggtatgagaaccacaacatctccattacaaatgccacagttgaagacagtggaacctactactgtacgggcaaagtgtggcagctggactatgagtctgagcccctcaacattactgtaataaaagct